(19) United States
(12) Patent Application Publication
(10) Pub. No.: US 2015/0038761 A1
(43) Pub. Date: Feb. 5, 2015

(54) GRAPHENE-AU NANOPLATE STRUCTURE, METHOD FOR FABRICATING THE SAME, AND METHOD FOR ACCELERATING CARBON IONS USING THE GRAPHENE-AU NANOPLATE STRUCTURE

(71) Applicant: Electronics and Telecommunication Research Institute, Daejeon (KR)

(72) Inventors: Wan-Joong KIM, Daejeon (KR); Moon-Youn JUNG, Daejeon (KR)

(73) Assignee: Electronics and Telecommunications Research Institute, Daejeon (KR)

(21) Appl. No.: 14/209,764

(22) Filed: Mar. 13, 2014

(30) Foreign Application Priority Data
Jul. 31, 2013 (KR) 10-2013-0091190

(51) Int. Cl. A61N 5/10

(52) U.S. Cl. CPC A61N 5/10; A61N 5/1077; A61N 2005/1088
USPC 600/1; 428/403; 427/2.1

(57) ABSTRACT

A graphene-Au nanoplate structure, a method for fabricating the same, and a method for accelerating carbon ions using the graphene-Au nanoplate structure. The graphene-Au nanoplate structure includes: a substrate; an Au nanoplate attached on the substrate; and a graphene wrapped around the Au nanoplate.

### CROSS-REFERENCE TO RELATED APPLICATION(S)

This application claims priority from Korean Patent Application No. 10-2013-0091190, filed on Jul. 31, 2013, in the Korean Intellectual Property Office, the disclosure of which is incorporated herein by reference in its entirety.

### BACKGROUND

1. Field

The following description relates to nano-structure fabrication and ion acceleration.

2. Description of the Related Art

Treatment technologies that can vaporize lesion tissues, such as cancer tissues, by injecting ions into target lesion tissues or cells, with reduced radiation exposure of normal cells, have been developed. For example, a proton therapy has been widely in use. In proton therapy, protons are focused around a lesion tissue of a patient by use of an acceleration device outside of the human body. When positively charged proton particles penetrate a body of a patient at a particular velocity, they decrease in velocity in the direction in which they are moving, by virtue of the electrostatic attraction between the proton particles and electrons in the body of the patient. Most proton particles move to a predetermined depth of the body until the velocity thereof becomes zero. A position where the most protons are stopped and accumulated is referred to as “Bragg peak”. A proton density in a non-Bragg peak area does not have any biological impact on cells. In X-ray treatments or gamma-ray treatments, most photons of X-rays or gamma rays are absorbed before they reach a target lesion tissue. As a result, side effects may occur which can transform normal cells to cancer (malignant or tumor) cells due to the radiation exposure of the normal cells.

Recently, ion beam treatments using carbon ions have been developed. Carbon ions allow sharper Bragg peak than proton ions. The carbon ion beam treatments allow for an increase in the density of carbon ions in the vicinity of Bragg peak and a decrease in the density of carbon ions in the normal cells, thereby reducing the radiation exposure of the normal cells in contrast to the proton treatments.

### SUMMARY

The following description provides an Au nanoplate structure including an Au nanoplate wrapped in graphene for accelerating carbon ions using a low-power laser, instead of a high-power laser, a method for fabricating the Au nanoplate structure, and a method for accelerating carbon ions using the Au nanoplate structure.

In one general aspect, there is provided a graphene-Au nanoplate structure including: a substrate; an Au nanoplate attached to the substrate; and a graphene wrapped around the Au nanoplate.

In another general aspect, there is provided a method for fabricating a graphene-Au nanoplate using an apparatus for fabricating a graphene-Au nanoplate, the method including: preparing a substrate; and attaching an Au nanoplate, wrapped in graphene, to the substrate.

In yet another general aspect, there is provided a method for accelerating carbon ions, the method including: preparing a graphene-Au plate structure comprising an Au nanoplate wrapped in graphene; generating accelerated carbon ions by emitting light to the graphene-Au nanoplate structure; and localizing the generated carbon ions into a target.

Other features and aspects will be apparent from the following detailed description, the drawings, and the claims.

### BRIEF DESCRIPTION OF THE DRAWINGS

FIG. 1 is a diagram illustrating an example scenario of an ion acceleration method using a graphene-Au nanoplate structure according to an exemplary embodiment.

FIG. 2 is a diagram illustrating an example of a graphene-Au nanoplate structure according to an exemplary embodiment.

FIG. 3 is a flowchart illustrating a method for fabricating a graphene-Au nanoplate structure according to an exemplary embodiment.

FIG. 4 is a diagram illustrating examples of a graphene-Au nanoplate structure using the method for fabricating a graphene-Au nanoplate structure illustrated in FIG. 3, according to an exemplary embodiment.

FIG. 5 is a transmission electron microscope (TEM) image of a graphene-Au nanoplate structure in accordance with an exemplary embodiment.

FIG. 6 is a flowchart illustrating a method for accelerating carbon ions using graphene according to an exemplary embodiment.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

Exemplary embodiments will now be described more fully hereinafter with reference to the accompanying drawings, in which exemplary embodiments are shown. The present disclosure may, however, be embodied in many different forms and should not be construed as limited to the exemplary embodiments set forth herein. Rather, these exemplary embodiments are provided so that the present disclosure is thorough, and will fully convey the scope of the invention to those skilled in the art.

Throughout the drawings and the detailed description, unless otherwise described, the same drawing reference numerals are understood to refer to the same elements, features, and structures. The relative size and depiction of these elements may be exaggerated for clarity, illustration, and convenience.

According to exemplary embodiments of the following description, an Au nanoplate structure comprising an Au nanoplate wrapped in graphene, a method for fabricating the Au nanoplate structure, and a method for accelerating carbon ions using the Au nanoplate structure are provided, which are applicable to cancer treatment in which light, such as, laser light is emitted to a target to induce carbon ions, and the carbon ions penetrate the human body to vaporize tumor cells. The purpose of the description is to enrich carbon ions using low-power laser light, instead of costly high-power laser light. Here, a structure in which an Au nanoplate is wrapped in graphene is referred to as a “graphene-Au nanoplate structure”. The graphene-Au nanoplate structure is a target to which a laser is radiated. The concept of “enrichment” is classified into the same category as “acceleration,” and thus both terms “enrichment (enrich)” and “acceleration (accelerate)” are used herein interchangeably. Cancer treatment is only taken as one example of the application of the description, and thus aspects of the exemplary embodiments may not be limited thereto. For convenience of description, the exemplary embodiments are provided with a focus on cancer treatment.

One ion acceleration method is to accelerate ions using a synchrotron or a cyclotron, which is not cost-effective. To implement this method, a large amount of costs are incurred, including construction costs for a three-story building, cost for setting up a system-wide shield, and maintenance cost.

The following description provides a method for accelerating ions by focusing a laser beam onto a proton target. This method is much more economical than the existing method of using a synchrotron or a cyclotron. When a laser beam is focused onto an ion generation target, ions are accelerated by a target normal sheath acceleration (TNSA) model or a radiation pressure acceleration (RPA) model. According to this method, a system occupies a relatively small space, and needs only a small area for a radiation shield, so that it is possible to economically operate the system with a small annual maintenance cost.

For cancer treatment using laser-induced ion acceleration, ions need to conform to the following two characteristics: the ions should be at high energy state to penetrate deep into the human body; and the ions should all have the same energy. For example, a proton of energy of 250 MeV penetrates the human body to a depth of 20 cm. Eye cancer treatment requires ions of high energy of about 70 MeV, and a treatment for cancer located deep in the body requires ions of much higher energy of 200 MeV or greater.

To generate such high energy ions, a high-power laser is needed, which is very costly. The following description provides, therefore, a graphene-Au nanoplate structure and a method for fabricating the graphene-Au nanoplate structure capable of generating high carbon ion energy, which is sufficient for use in cancer treatment using a low-power laser, rather than a high-power laser.

Hereinafter, a graphene-Au nanoplate structure, a method for fabricating the same, and a method for accelerating carbon ions using the graphene-Au nanoplate structure will be described in detail with reference to accompanying drawings.

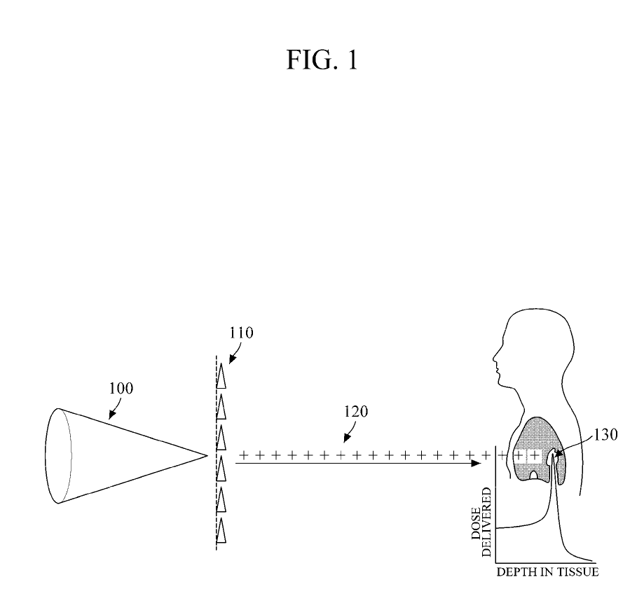

FIG. 1 is a diagram illustrating an example scenario of an ion acceleration method using a graphene-Au nanoplate structure according to an exemplary embodiment.

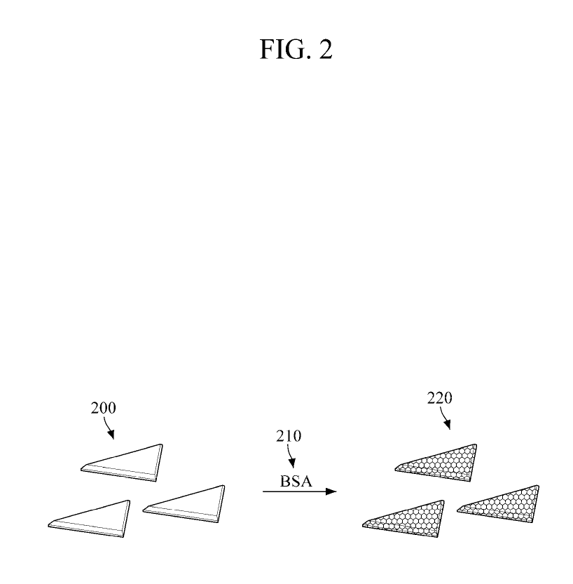

Referring to FIG. 1, carbon ions that are accelerated by the graphene-Au nanoplate structure penetrate the human body to a tumor 130 and are localized in the tumor 130. As shown in FIG. 1, when a laser beam 100 is radiated to the graphene-Au nanoplate structure 110 arranged at the focal distance of the laser beam, carbon ions 120 are accelerated and localized in the tumor 130 in the body of a patient. The form of the graphene-Au nanoplate structure will be described with reference to FIG. 2.

A position of the tumor 130 at which the ions are dense is the same as a position of Bragg's peak. Position of Bragg's peak refers to a position at which a velocity of a charged particle that moves through a matter becomes zero due to energy loss caused by opposite charges in the matter. Once location information of the tumor 130 has been identified based on X-ray images or MRI images, the intensity of energy of particles may be adjusted, in advance, using the identified location information, such that ions can precisely localized in the tumor 130, as shown in FIG. 1. In this example, the graphene-Au nanoplate structure 110 is used as a source to accelerate carbon ions using a low-power laser. FIG. 2 is a diagram illustrating an example of a graphene-Au nanoplate structure according to an exemplary embodiment.

Referring to FIG. 2, the graphene-Au nanoplate structure 220 is fabricated by wrapping graphene around an Au nanoplate 200.

The Au nanoplate 200 may have at least one pointed end. For example, as shown in FIG. 2, the Au nanoplate 200 may have a triangular shape. Alternatively, the Au nanoplate 200 may consist of Au nanoparticles. In another example, the Au nanoplate 200 may include a plurality of Au nanoplates arranged at a predetermined interval. The predetermined interval may be about 20 nm. However, the embodiment is not limited thereto. In another example, the Au nanoplate 200 may include a plurality of Au nanoplates arranged in a bow tie form. The Au nanoplates are arranged such that a plasmon phenomenon occurs in the graphene-Au nanoplate structure 220 to induce a more intensified optical electromagnetic field.

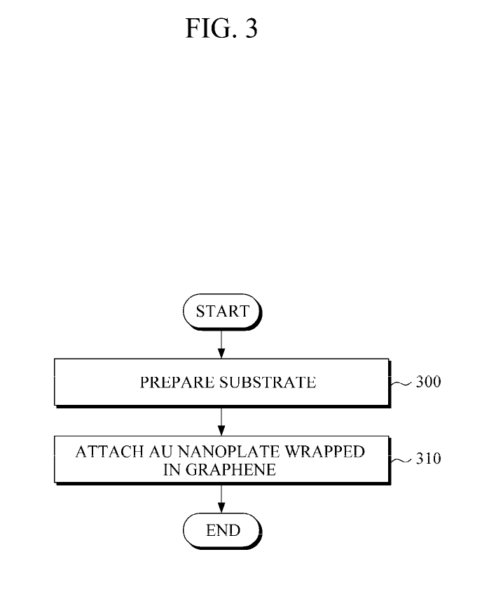

In one example, for the graphene-Au nanoplate structure 220, bovine serum albumin (BSA) 200 is used to wrap graphene around the Au nanoplate 200. BSA 210 is a hydrophilic protein with an amine group (—NH₂), which is easily coupled to the Au nanoplate 200. In addition, the amine group (—NH₂) of BSA 200 facilitates an ionic bonding with a carboxyl group (—COOH) of the graphene. As a result, it is possible to produce a graphene-COO-NH3+-albumin. A method for fabricating the graphene-Au nanoplate structure 220 using BSA 210 is described in detail with reference to FIG. 3.

FIG. 3 is a flowchart illustrating a method for fabricating a graphene-Au nanoplate structure according to an exemplary embodiment.

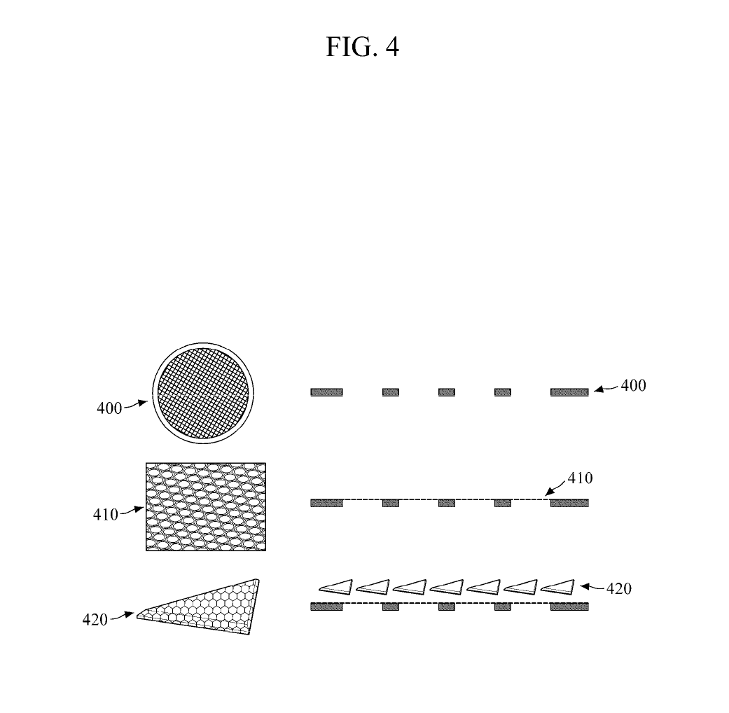

Referring to FIG. 3, a substrate is prepared in 300. The substrate acts as a support layer. As described later with reference to FIG. 4, the substrate may be in the form of a transmission electron microscope (TEM) grid.

In 310, a graphene-Au nanoplate is attached on the substrate. The graphene-Au nanoplate may be formed of sodium citrate and poly vinyl pyrrolidone (PVP).

In one example, a graphene-Au nanoplate structure may be formed by wrapping graphene around an Au nanoplate using BSA. Here, the example method for fabricating the graphene-Au nanoplate structure using BSA is described in detail.

1. 10% BAS (100 μl) is mixed with an Au nanoplate in a 1 ml Ep tube, and a mixture is agitated for 1 hour.

2. After 1 hour, remaining BSA is removed by centrifugation at 12,000 rpm for 20 minutes.

3. 1 ml of distilled water is added, and still remaining BSA is removed by centrifugation.

4. 1 ml of distilled water is further added, to which 0.1 mg/ml graphene is added and the mixture is agitated.

5. After 2 hours of agitation, remaining graphene is removed by centrifugation at 12,000 rpm for 20 minutes.

The aforesaid method for fabricating a graphene-Au nanoplate structure is given only for purpose of example, and equipment and the amount of components may vary depending on the fabrication conditions.

FIG. 4 is a diagram illustrating examples of a graphene-Au nanoplate structure using the method for fabricating a graphene-Au nanoplate structure illustrated in FIG. 3, according to an exemplary embodiment.

Referring to FIG. 4, the graphene-Au nanoplate is in a TEM grid. Reference numeral 400 on the left side of FIG. 4 represents a top view of a grid as a support layer, and reference numeral 400 on the right side of FIG. 4 illustrates a cross-sectional view of the grid. In 400 on the left side of FIG. 4, dotted lines represent a mesh form of the grid.

Graphene 410 is attached onto the grid 400. Top view 410 on the left side of FIG. 4 does not show the grid 400 beneath the graphene 410. Cross-sectional view 410 on the left side of FIG. 4 shows the graphene 410 attached onto the grid 400. In so doing, a graphene layer 410 is formed on the grid 400 as a support layer. The above process of attaching the graphene 410 onto the grid 400 can be omitted.

Then, the graphene-Au nanoplate 420 as fabricated through the processes described with reference to FIG. 3 is situated on the substrate 400.

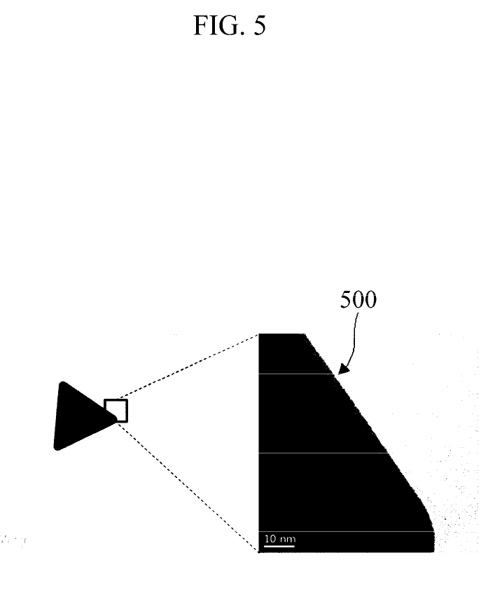

FIG. 5 is a TEM image of a graphene-Au nanoplate structure in accordance with an exemplary embodiment.

Referring to FIG. 5, graphene 500 wraps around an Au nanoplate.

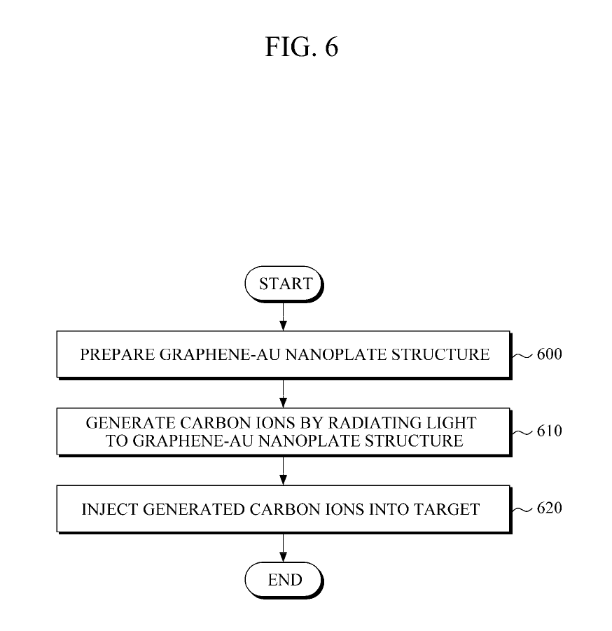

FIG. 6 is a flowchart illustrating a method for accelerating carbon ions using graphene according to an exemplary embodiment.

Referring to FIG. 6, a graphene-Au nanoplate structure is prepared in 600. In 610, light is emitted to the graphene-Au nanoplate to generate carbon ions. The light emitted may be laser light, but the type of light is not limited thereto.

In 610, a surface plasmon phenomenon occurs in the vicinity of a graphene-Au nanoplate, due to the graphene-Au nanoplate structure irradiated by the light, thereby accelerating energy intensity. The accelerated energy ionizes carbon atoms within the graphene in the graphene-Au nanoplate structure, and carbon ions accelerating toward a target are generated. The target may be human lesion tissue, and specifically, cancer tissue, but the type of target is not limited thereto. In 620, the carbon ions are localized to the target.

According to the exemplary embodiments, when a laser beam is emitted into the graphene-Au nanoplate structure, a plasmon phenomenon occurs in an Au nanoplate, which causes an intensity of the incident laser beam to be increased 5 to 100 times higher, so that it is possible to obtain high-energy carbon ions. In addition, it is possible to accelerate carbon ions using a low-power laser, instead of a high-power laser.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A graphene-Au nanoplate structure comprising:
a substrate;
an Au nanoplate attached to the substrate; and
a graphene wrapped around the Au nanoplate.

2. The graphene-Au nanoplate structure of claim 1, wherein the substrate is in the form of a transmission electron microscope (TEM) grid.

3. The graphene-Au nanoplate structure of claim 1, wherein the Au nanoplate has at least one pointed end.

4. The graphene-Au nanoplate structure of claim 3, wherein the Au nanoplate has a triangular shape.

5. The graphene-Au nanoplate structure of claim 1, wherein the Au nanoplate comprises gold (Au) nanoparticles.

6. The graphene-Au nanoplate structure of claim 1, wherein the Au nanoplate comprises a plurality of Au nanoplates arranged at a predetermined interval.

7. The graphene-Au nanoplate structure of claim 6, wherein the plurality of Au nanoplates are arranged in a bow tie form.

8. A method for fabricating a graphene-Au nanoplate using an apparatus for fabricating a graphene-Au nanoplate, the method comprising:
preparing a substrate; and
attaching an Au nanoplate, wrapped in graphene, to the substrate.

9. The method of claim 8, wherein the attaching of the Au plate, wrapped in graphene, to the substrate comprises fabricating the Au nanoplate; and wrapping the graphene around the Au nanoplate.

10. The method of claim 10, wherein the fabricating of the Au nanoplate comprises fabricating the Au nanoplate using sodium citrate and poly vinyl pyrrolidone (PVP).

11. The method of claim 9, wherein the wrapping of the graphene around the Au nanoplate comprises wrapping the graphene around the Au nanoplate using bovine serum albumin (BSA).

12. The method of claim 11, wherein the BSA has hydrophilic properties, and comprises an amine group (-NH₂) which is easily coupled to the Au nanoplate and facilitates ionic bonding with a carboxyl group (—COOH) of the graphene.

13. The method of claim 11, wherein the wrapping of the graphene around the Au nanoplate comprises inserting the Au nanoplate into a tube; injecting the BSA into the tube containing the Au nanoplate, and agitating a resulting mixture in the tube; removing remaining BSA using centrifuge; further removing remaining BSA after the centrifuge, using distilled water; adding distilled water and graphene into the tube from which the BSA has completely been removed, and agitating a mixture in the tube; and removing graphene after agitation using the centrifuge.

14. The method of claim 8, further comprising:
attaching graphene onto the substrate,
wherein the attaching of the Au nanoplate wrapped in graphene comprises attaching the Au nanoplate wrapped in graphene onto the substrate onto which the graphene has been attached.

15. A method for accelerating carbon ions, the method comprising:
preparing a graphene-Au plate structure comprising an Au nanoplate wrapped in graphene;
generating accelerated carbon ions by emitting light to the graphene-Au nanoplate structure; and
localizing the generated carbon ions into a target.

16. The method of claim 15, wherein the generating of the accelerated carbon ions comprises accelerating energy through a surface plasmon phenomenon which occurs in the vicinity of the graphene-Au nanoplate by the light emitted to the graphene-Au nanoplate structure; and generating carbon ions accelerated to the target by ionizing carbon atoms within the graphene-Au nanoplate structure through the accelerated energy.

17. The method of claim 15, wherein the injecting of the carbon ions comprises, in response to irradiating the graphene-Au nanoplate structure disposed at a focal length with light, causing the carbon ions accelerated within the graphene-Au nanoplate structure to be accelerated.

18. The method of claim 15, wherein the light is laser light.

19. The method of claim 15, wherein the target is human lesion tissue.
